(19) United States
(12) Patent Application Publication
(10) Pub. No.: US 2022/0296105 A1
(43) Pub. Date: Sep. 22, 2022

(54) PHOTOPLETHYSMOGRAPHY DERIVED BLOOD PRESSURE MEASUREMENT CAPABILITY

(71) Applicant: Nicholas D.P. Drakos, Southern Pines, NC (US)

(72) Inventor: Nicholas D.P. Drakos, Southern Pines, NC (US)

(21) Appl. No.: 17/655,902

(22) Filed: Mar. 22, 2022

Related U.S. Application Data
(60) Provisional application No. 63/164,073, filed on Mar. 22, 2021.

(51) Int. Cl. A61B 5/022; A61B 5/024; A61B 5/00; A61B 5/0215; A61B 5/1455

(52) U.S. Cl. CPC A61B 5/022; A61B 5/02416; A61B 5/7221; A61B 5/7264; A61B 5/0215; A61B 5/14551; A61B 5/7203; A61B 5/6801

(57) ABSTRACT

A photoplethysmography (PPG) signal is used together with a neural network processing model to obtain convenient, timely, and reliable blood pressure measurements. The method, system, and device manifestation of the invention generally involves development of a neural network model for deriving blood pressure from a PPG signal and deployment of the model as blood pressure measurement capability. Development (500) of the neural network model includes: data capture (502 and 504) involving both PPG signal data and “ground truth” blood pressure data, optionally with subject demographic and health data (506); data processing (508); neural network model development (510); and neural network model validation (512). The neural network model can then be deployed as a blood pressure measurement capability for intermittent or continuous blood pressure measurement.

### RELATED APPLICATION INFORMATION

This application claims the benefit of U.S. Provisional Patent Application No. 63/164,073 entitled “Photoplethysmography Derived Blood Measurement Capability,” filed Mar. 22, 2021, the content of which is incorporated herein by reference in full and priority from that application is claimed to the full extent allowed by U.S. law.

### FIELD OF THE INVENTION

The present invention relates to medical diagnosis, evaluation, and monitoring of human blood pressure in healthcare and non-healthcare settings by experts and non-experts and, in particular, to a method, system, and device manifestation for obtaining rapid intermittent or continuous blood pressure information for medical diagnostic or monitoring purposes or for tracking general health and wellness metrics.

### BACKGROUND OF INVENTION

Blood pressure is one of the core vital signs for evaluating human health, both in acute and chronic settings. In the acute setting, blood pressure that is above or below the normal range can either precipitate or be the consequence of a range of serious and life-threatening medical conditions. High blood pressure is associated with acute coronary syndromes (ACS), stroke, aortic dissection, kidney failure, and multiple other serious acute conditions. Low blood pressure is associated with shock, sepsis, traumatic hemorrhage, dehydration, and multiple other life-threatening conditions. As such, blood pressure plays a critical role in risk-stratifying patients, diagnosing acute illness and injury, and in guiding treatment. Normal blood pressure also plays an important role in the acute setting as an indicator of the absence of a serious condition requiring immediate evaluation and treatment and is a key part of triage and risk-stratification in settings such as the emergency department.

Chronic hypertension, or chronically elevated blood pressure, is one of the most prevalent and consequential medical conditions in the world today. As of 2019, the World Health Organization estimated that 1.13 billion people suffer from hypertension and identified hypertension as one of the leading causes of premature death worldwide. Thus, the ability for both medical personnel and lay persons to acquire human blood pressure measurements effortlessly, swiftly, and accurately has significant implications for human health, healthcare related costs, and systemic healthcare efficiencies.

Blood pressure measurement techniques are broadly classified as non-invasive versus invasive and intermittent versus continuous. The most common methodologies are intermittent non-invasive and continuous invasive. Continuous non-invasive techniques currently exist but are much less common. Intermittent non-invasive measurements, which is used here to describe acquisition of a single blood pressure measurement at a single point in time or multiple single blood pressure measurements collected over time, most commonly use a sphygmomanometer, a pneumatic cuff device that was first invented in the 1880's. This technique involves inflating a cuff, commonly around the upper arm, until the underlying artery is occluded. Blood pressure is then determined as the pressure is released and blood flow resumes in the underlying artery at both a maximal (systolic) and minimal (diastolic) pressure. This method is widely used in healthcare and home settings. Contemporary automated blood pressure cuffs typically function using the oscillometric method.

The pneumatic blood pressure cuff method for the determination of blood pressure is generally safe and effective but has several drawbacks. It can be difficult and cumbersome to self-apply a blood pressure cuff, which can affect the accuracy of measurements or even the frequency at which people will monitor their blood pressure. This may be particularly true for the population that is most likely to have hypertension and require home monitoring, such as the elderly or those with other disabilities or medical conditions. In addition, the high inflation pressure of the cuff can cause discomfort. In healthcare settings, the same blood pressure cuff will frequently be used for multiple patients. Given the large surface area and inflation hoses, which may touch the floor, it represents a potential vector for disease transmission across patients. Those same hoses may also present a trip and fall hazard for patients.

There are also time and efficiency considerations with sphygmomanometer blood pressure cuff application and use, from a time standpoint it is often the rate limiting step in obtaining routine vital signs. Routine vital signs consist of temperature, heart rate, blood pressure, respiratory rate, and arterial oxygen saturation. Obtaining blood pressure via a blood pressure cuff requires an additional one to two minutes beyond the acquisition of the other routine vital signs. Consider that these one to two minutes multiplied across approximately 150 million annual US emergency department visits equate to 2.5-5 million hours or 285-570 years of time dedicated just to acquiring initial triage vital signs on US emergency department patients each year. Based on this consideration, the ability to acquire a non-noninvasive blood pressure more rapidly has significant implications for cost and efficiency in US healthcare.

Another common blood pressure acquisition method, primarily used in an intensive care unit (ICU) setting, is continuous invasive blood pressure monitoring. This is accomplished by inserting a catheter into an artery (typically at the wrist but arteries at multiple sites are feasible) to directly measure blood pressure within the arterial component of the cardiovascular system. The advantages to this method are the accuracy and the ability to acquire a continuous blood pressure tracing. However, the invasive nature of the technique has multiple associated risks including pain, infection, and damage to the canulated artery and/or other internal structures.

Lastly, continuous non-invasive blood pressure acquisition methods exist. However, they have not been as widely adopted as the methods described above. While this method has a number of advantages, the lack of adoption may be due to concerns regarding accuracy and additional hardware requirements that diminish advantages over the other methods described above.

### SUMMARY OF INVENTION

The present invention is directed to an evaluation system and associated functionality for the non-invasive intermittent and continuous measurement of human blood pressure that is useful for rapid, facile, and low-risk blood pressure determination in multiple environments and contexts including healthcare settings, out-of-hospital, and emergency settings, for home use and general health monitoring by non-medical experts, and for use in telemedicine applications. In emergency settings, particularly involving time constrained critical illness and injury (TCCI) where the probability of favorable outcomes is directly related to timely intervention, this invention potentiates rapid and low risk acquisition of blood pressure information. This is key information for medical providers to attain a diagnostic certainty threshold for intervention to mitigate or avert the underlying medical risk; for TCCI, the earlier that a diagnostic certainty threshold is reached and the earlier an appropriate intervention is accomplished the higher the probability of a favorable outcome. On the other end of the spectrum, this intervention potentiates blood pressure monitoring compliance by individuals with conditions such as hypertension and diabetes who benefit from frequent blood pressure monitoring at home. These individuals traditionally use some version of sphygmomanometry, which can be cumbersome to apply correctly, can be painful, and only provides intermittent results. This invention provides a simpler, pain free intermittent or continuous blood pressure monitoring capability with comparable accuracy. There are multiple use cases, in both healthcare and non-healthcare settings, in addition to those described above where this invention provides significant utility over common existing blood pressure acquisition methods through rapid and facile blood pressure acquisition with less time, cost, and risk.

In accordance with one aspect of the present invention, a method and apparatus (“utility”) are provided for measuring blood pressure based on signals from a photoplethysmography (PPG) device and another blood pressure measurement device. The utility involves obtaining first and second processed signal information and developing a neural network model for obtaining blood pressure information using the first and second processed signal information. The first signal information corresponds to first signals of one or more first subjects obtained using a PPG device. The second signal information corresponds to second signals obtained using a blood pressure device different than the PPG device. The neural network model is developed based on signals of the PPG device using the first and second processed signal information. The utility further involves validating the neural network model by comparison of blood pressure measurements obtained using the model blood pressure measurements obtained using one or more other blood pressure measurement devices. The invention thus enables blood pressure measurements based on a signal from a PPG device with an accuracy that is improved by model verification in relation to another blood pressure device such as a gold standard or ground truth device for blood pressure measurement.

In certain implementations, the first signals are obtained using the PPG device on the first subjects and the second signals are obtained by using the blood pressure device on the same first subjects. The PPG device may be a purpose-built pulse oximetry device, a wearable health device, or a smart phone. The blood pressure device may be a pneumatic cuff device or an invasive, continuous blood pressure measuring device. The first and second signals may be preprocessed to get the first and second processed signal information. For example, such preprocessing may involve feature identification and extraction and/or time shifting at least one of the first and second signals for alignment of waveforms.

In accordance with another aspect of the present invention, a utility is provided for determining blood pressure based on a PPG signal and a neural network. The utility involves providing a neural network model for obtaining blood pressure information, operating a PPG device to obtain a signal from a first subject, preprocessing the signal to obtain preprocessed signal information, and applying the neural network model to the preprocessed signal information to obtain blood pressure information. The resulting blood pressure information can then be provided to a user such as a medical professional or layperson.

In certain implementations, the neural network model may be used to define an application program interface (API) to facilitate remote acquisition of the blood pressure information. For example, PPG signal information may be uploaded from a smart phone or other data terminal to a local or remote platform implementing the neural network model. The neural network model can then be used to determine blood pressure information based on the PPG signal and to provide such information to the data terminal or a different data terminal. The neural network may also ingest blood pressure information from a different blood pressure device for purposes of, for example, neural network model development and deployment.

In accordance with a further aspect of the present invention, a utility is provided for processing of PPG information to obtain blood pressure information. The utility comprises a storage unit, and input module, and a processing platform. These storage unit stores a neural network model for obtaining blood pressure information based on signals from a PPG device. The input module is operative for receiving, from the PPG device, a signal for a first subject. The processing platform is operative for preprocessing the signal to obtain preprocessed signal information for use in the neural network model, applying the neural network model to the preprocessed signal information to obtain blood pressure information, and outputting the blood pressure information. Again, the neural network may further ingest blood pressure information from a different blood pressure device for purposes of neural network model development and deployment.

In accordance with a still further aspect of the present invention, a utility is provided for obtaining PPG sensor unit information for use in determining blood pressure information. The utility involves a sensor unit and a processor. The sensor unit is operative for noninvasively obtaining a PPG signal for a subject. The processor is operative for receiving signal information based on the PPG signal and formatting a blood pressure request based on the signal information for transmission to a processing platform via an API, where the API is defined based on a neural network model for obtaining blood pressure information based on PPG signals. The processor is further operative for transmitting the blood pressure request to the processing platform, receiving blood pressure information from the processing platform, and displaying the blood pressure information.

### BRIEF DESCRIPTION OF THE DRAWINGS

For a more complete understanding of the present invention, and further advantages thereof, reference is now made to the following detailed description taken in conjunction with the drawings, in which:

FIG. 1 is a schematic diagram of a risk stratification and medical diagnosis system in accordance with the present invention showing a first use case related to field use outside of a medical facility;

FIG. 2 is a schematic diagram of a risk stratification and medical diagnosis system in accordance with the present invention showing a second use case related to use within a medical facility;

FIGS. 3A-3B show schematic diagrams illustrating operation of a processing system of a risk stratification and medical diagnosis system in accordance with the present invention for data collection, correlation and model training;

FIGS. 4A-4B show schematic diagrams illustrating operation of a processing system of a risk stratification medical diagnosis system in accordance with the present invention for model deployment; and

FIGS. 5A-5B show schematic diagrams illustrating operation of a processing platform of a blood pressure measurement system in accordance with the present invention.

### DETAILED DESCRIPTION

The present invention relates to using a photoplethysmography (PPG) signal together with a neural network processing model to obtain convenient, timely, and reliable blood pressure measurements. In the following description, the invention is set forth in the context of specific neural network models involving a second blood pressure device and associated system implementations and use cases. While these are believed to represent advantageous implementations and provide a fulsome understanding of the invention, the invention is not limited to such implementations. Accordingly, the following description should be understood as illustrative and not by way of limitation.

The following discussion first provides a description of the PPG-based blood pressure capability and associated processing. Thereafter, various system implementations are described for emergency and non-emergency applications, including in healthcare facilities and outside of healthcare facilities and involving medical professional users and laypeople.

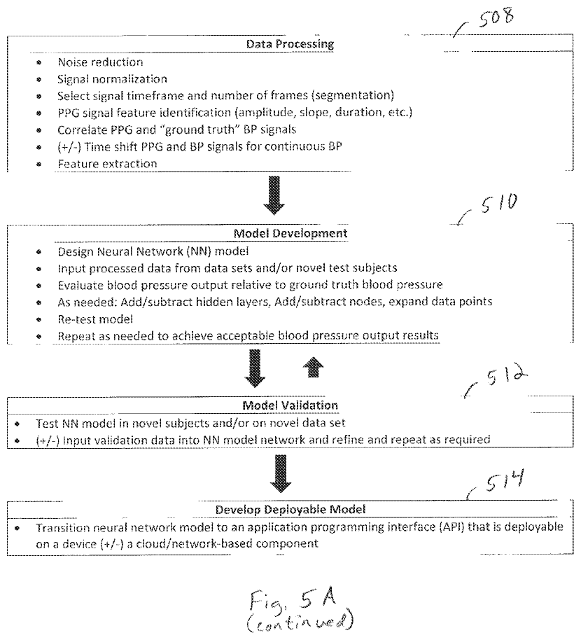

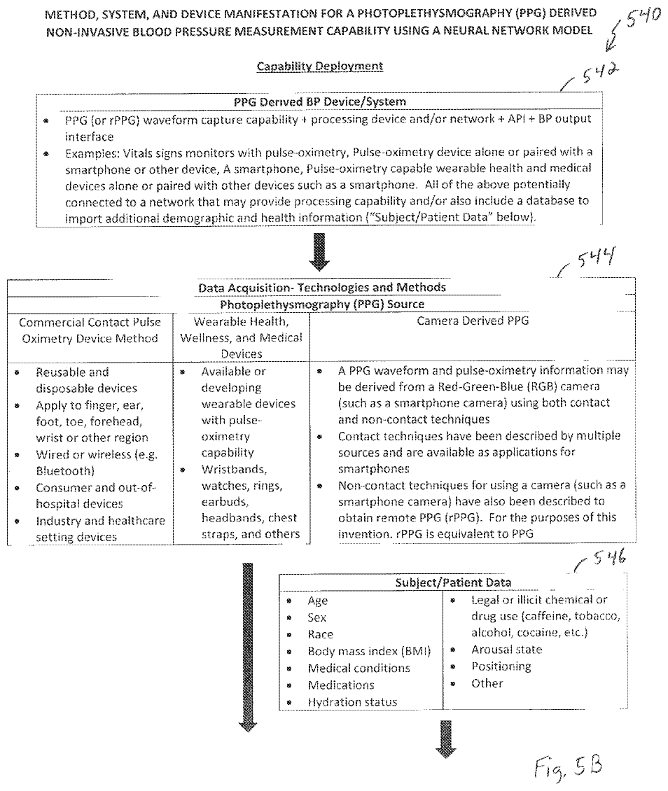

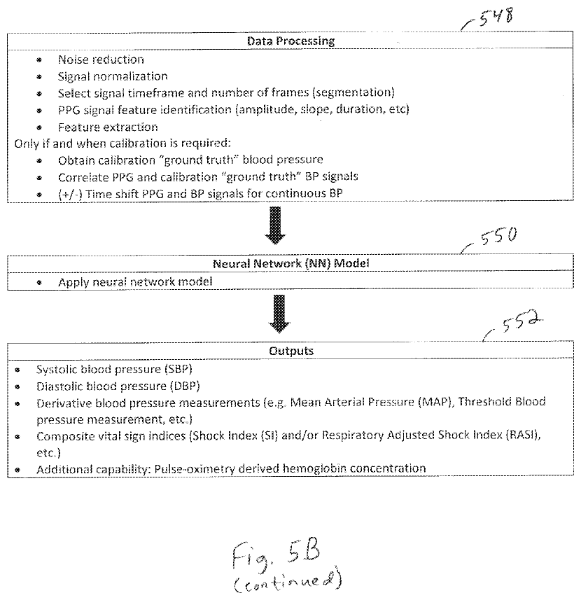

The method, system, and device manifestation of the invention generally involves development (FIG. 5A) of a neural network model for deriving blood pressure from a PPG signal and deployment (FIG. 5B) of the model as blood pressure measurement capability.

Development (500) of the neural network model involves the following general steps (FIG. 5A): 1) Data Capture (502 and 504): PPG signal data and “ground truth” blood pressure data (504)+/−subject demographic and health data (506) 2) Data Processing (508), 3) Neural Network Model development (510), and 4) Neural Network Model validation (512). Deployment (540) of the neural network model as a blood pressure measurement capability involves the following general steps (FIG. 5B): 1) Conversion of the Neural Network Model to an application programing interface (API) and placement of the API on a system such as a device and/or network (542), 2) Capture PPG data (544)+/−demographic and health data (546), 3) Data Processing (548), 4) Applying the neural network model (550) to processed PPG data via API, and 5) Outputting (552) intermittent or continuous blood pressure measurement.

For either development (500) or deployment (540) of the invention, PPG data can be captured via multiple methods, techniques, and devices. These methods and techniques include, but are not limited to, purpose-built disposable and reusable pulse-oximetry monitors and devices; wearable health and medical devices with pulse-oximetry capability such as Apple Watch, Fitbit, Garmin, Wellue Rings and others; applications for acquiring PPG and pulse-oximetry information by placing a finger over the light source and camera on a smartphone or similar device; and the use of red-green-blue (RGB) cameras, such as on smartphones, and processing and analytic capability to capture and display remote photoplethysmography (rPPG) waveforms. Furthermore, PPG waveforms can be captured for the purposes of this invention via transmissive or reflectance pulse-oximetry techniques. From the standpoint of this invention, it does not matter how the PPG (or rPPG) waveform is obtained as long as it reflects arterial pulsation. For the development of the neural network model underlying the capability, both PPG and blood pressure data may be, in whole or part, acquired and input from existing data sets (502).

For the development of the neural network model underlying this invention, “ground truth” blood pressure measurements may be obtained on the same group of subjects from whom PPG data has been acquired. Blood pressure data (504) can be acquired through different techniques which include, but are not limited to, intermittent non-invasive techniques, such as using a blood pressure cuff; continuous non-invasive techniques; or continuous invasive techniques, such as with an arterial line. For deployment of the model, blood pressure measurements may be required to calibrate the neural network model at the level of individual users and/or across groups of users. However, blood pressure data inputs will not be required with each use of the capability and may not be required at all.

Both development and deployment of the model may, but will not necessarily, also use data (506 and 546) such as age, sex, body mass index (BMI), race, medications, hydration status, the use of alcohol, tobacco, caffeine, or other drugs or chemicals, known medical conditions such as, but not limited to, hypertension, diabetes, or cardiovascular disease to further improve the accuracy and predictive capability of the neural network model. It may further incorporate such data as the patient's or subject's current state of arousal—relaxed, anxious, fearful, just woke up, just exercised, etc.—and the patient's current body position—laying, sitting, standing, and for how long. This information may be manually input into the network, system, or device running the model or it may be retrieved automatically from sources such as wearable health devices and/or through network capabilities from electronic health records (EHR), or other sources.

For development of the neural network model, PPG, blood pressure (BP), and any other data will undergo processing (508) to include, but not limited to, noise reduction, normalization, segmentation, feature identification and extraction, and, where continuous blood pressure waveforms are used, time shifting of PPG and BP waveforms to align specific features in time. For deployment of the neural network model and capability, blood pressure data will only be input into the model if and when calibration is required and, in those instances, processing (548) will be required for blood pressure data as described above.

For development of the neural network model, processed data will be input into the neural network (510). The neural network will output blood pressure as systolic blood pressure and diastolic blood pressure. It will also be capable of providing a mean arterial pressure (MAP) and other derivative blood pressure measurements and indices using pulse rate and respiratory rate data that are acquired via pulse oximetry. Examples of output indices include, but are not limited to, Shock Index (SI) and/or Respiratory Adjusted Shock Index (RASI). The neural network will then be validated (512) on live subjects and/or models using both new PPG and ground truth blood pressure measurements. The results of this validation will further inform the neural network model. The process of cycling through neural network model development and validation continues until the blood pressure outputs from the model are within tolerance for contemporary blood pressure monitoring devices or until they are within tolerance for other blood pressure monitoring applications.

At this juncture, the neural network model (514) will be converted to an application programming interface (API) for deployment. The API will run on a device and/or network as part of a device and/or system (542) that will capture (544) a PPG waveform and other data inputs (546) noted above, process (548) the PPG waveform data and other data, apply the neural network model (550) to the input data, and output (552) a systolic blood pressure (SBP), diastolic blood pressure (DBP), and other derivative blood pressure measurements including, but not limited to, mean arterial pressure (MAP). The capability can also utilize blood pressure measurements in conjunction with other vital signs metrics commonly acquired by pulse oximetry, such as heart rate and respiratory rate, to calculate and output indices including Shock Index (HR/SBP) and/or Respiratory Adjusted Shock Index ((HR/SBP)×(RR/10)). The blood pressure measurement will display on a monitor that is part of the device and/or system that constitute the capability, such as the screen on a finger applied pulse-oximeter, a smartphone screen, a vital-signs monitor screen, etc. The blood pressure measurements may be output as intermittent or continuous readings. The neural network model may require occasional calibration for individual users of the invention or across populations who will be using the invention.

The optimal manifestation of this capability will output systolic and diastolic blood pressure information, within an acceptable tolerance of accuracy, via the analysis of an input PPG waveform by a neural network model without requiring calibration or the input of demographic information. This pulse-oximetry derived blood pressure measurement capability may also be combined into a single capability that includes other pulse-oximetry derived measurements including oxygen saturation, heart rate, respiratory rate, and hemoglobin concentration to provide an array of key physiologic and pathologic metrics for range of health conditions across a range of circumstances. For example, all of these outputs combined into a single capability would provide an ideal tool for triaging and monitoring patients in a mass casualty (MASCAL) event. It would allow healthcare providers to easily risk stratify patients based on such indices and metrics as Respiratory Adjusted Shock Index and hemoglobin trends to rapidly determine resource requirements for individual patients and across groups of patients.

The structure and functionality described above may be implemented in a system as described below, where one or more APIs may be provided to facilitate communications between applications running on the various platforms. Such an API, including the messaging, formats, and fields, may be defined based on the neural network model.

In the following description, the invention as set forth in certain contexts relating to use by a non-expert, or layperson, in an emergency environment and use by experts (e.g., doctors and other medical care providers) in a medical facility. In these cases, blood pressure may be just one parameter used by the system. However, it will be understood that a dedicated blood pressure measurement system may be implemented without the need for the extraneous elements as described below and may be implemented with local or remote processing. While these examples are useful in illustrating the flexibility of the invention, it will be appreciated that the invention is applicable in other contexts such as for use by first responders, use by combat medical personnel, use by staff medical personnel in schools, businesses, and other entities, and other environments involving nonexpert, semi-expert and expert users. Moreover, while the invention is described below for use in connection with certain examples of evaluating TCCI conditions, it will be appreciated that various aspects of the invention are more broadly applicable, including outside of medical contexts. Thus, the following description sets forth a number of examples relating to medical applications and then discusses a variety of other non-limiting use cases.

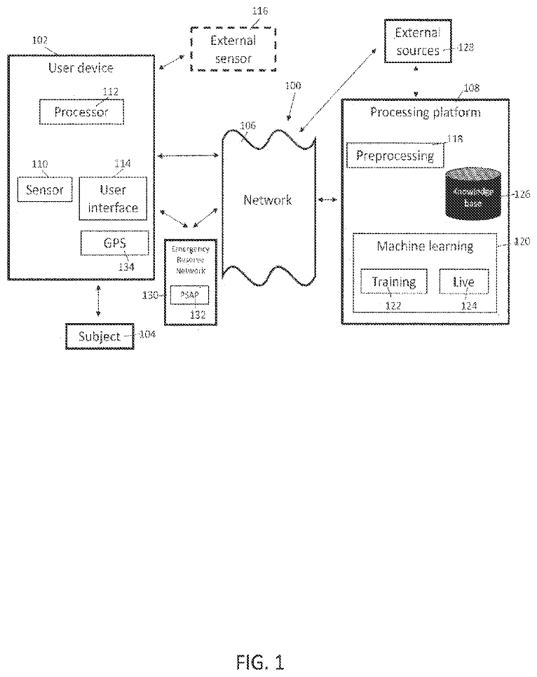

FIG. 1 is a schematic diagram of a Predictive Diagnostic Information Capability-Technology (PreDICT™) system 100 in accordance with the present invention. More specifically, FIG. 1 illustrates the system 100 in connection with a first use case relating to use of the system in a medical context and in the field, i.e., outside of a medical facility. Such use may be by a nonexpert users such as a layperson, by a first responder, or others. Moreover, data for the system 100 may be collected by medical providers, laypersons, users, subjects, or a third party not expressly for the purposes of the system. Data may be ingested and utilized for diagnosing and treating novel patients or it may be captured and compared against previously ingested data for a specific patient or group of patients. Previously ingested data may have been for the purposes of establishing a baseline or for the purposes of providing diagnosis and treatment or for another purpose altogether. However, for purposes of illustration, the illustrated system 100 generally includes a user device 102 for use by a user assisting a subject 104, a processing platform 108, and a network 106 for connecting the user device 102 to the processing platform 108. The system 100 may also involve an emergency response network 130 that includes public-safety answering points (PSAPs) 132 or similar network infrastructure in secure and unsecure, classified, and unclassified military, maritime, disaster or other communication networks.

The illustrated user device 102 may include, for example, a smart phone, tablet computer or similar device. The user device 102 includes one or more sensors 110, a processor 112, and a user interface 114. As will be understood from the description below, a variety of types of sensors may be utilized including, for example, the device's video camera, the device's touchscreen, a microphone, or the like. Optionally, external sensors 116 such as an infrared camera, a pulse oximetry sensor (e.g., used to obtain oxygen saturation information, pulse rate, or blood pressure as described above), a digital thermometer or the like may be used in conjunction with the user device. For example, such sensors may be incorporated into a wearable in communication with the user device. Information from other types of sensors, such as impact monitors implemented in helmets for sports or military use, may also be employed.

In alternate use cases, such as battlefield environments or applications that ingest information from drones, available security cameras, or other sources, different workflows may be involved, for example, not involving an interactive interface for data acquisition. In the illustrated use case, the user interface 114 can be used to access the processing platform, to input information about the subject or the condition at issue, to provide information about the location or environment or other information that may be useful by the processing platform 108. The user interface may be implemented via voice activation, a touchscreen, a keyboard, graphical user interface elements and the like. The functionality of the sensor 110 and user interface 114 may be executed on the processor 112. The processor 112 is also operative for executing a variety of input and output functions, for example, related to interfacing with the processing platform 108.

The system 100 may also use information regarding the location of the user device 102. Where the user device 102 includes a GPS module 134 or other location information provisioned by satellite constellations, such information may be reported to the processing platform or used to route first responders to the user device 102. In other cases, location information may be provisioned by a cellular network technology such as angle of arrival, time delay of arrival, cell ID, cell sector, microcell, or other location technologies. Such location information may be provided to the processing platform 108 and emergency response network 130 via the user device 102 or via a separate pathway, e.g., from a network location information gateway. Location data may also be derived from recognition by the technology of environmental signatures including, but not limited to, image and acoustic signatures at a specific location that serve to localize, at some level of specificity, where the technology is being applied.

The system 100 may be implemented via a variety of architectures. For example, the functionality described in more detail below may be cloud-based such that little or no logic is required on the user device 10 to the implement the functionality. Alternatively, an application may reside on the user device 102 to support all or certain functionality of the system 100. For example, certain preprocessing may be executed locally to support the machine learning functionality of the processing platform 108. As a still further alternative, some of the logic may be implemented within the emergency response network 130, for example, at a PSAP 132. Thus, for example, a layperson assisting a subject 104 in an emergency environment may dial an emergency phone number (e.g., 911 in the United States) via a telephony or data network (e.g., VOIP). In such cases, the emergency call may be routed to an appropriate PSAP 132 via conventional network processes. Emerging technologies allow files to be uploaded from the user device 102 to the PSAP 132, including video and audio files. Accordingly, sensor information and other information from the user device 102 can be routed to the PSAP 132 which may in turn interface with the processing platform 108 to implement the functionality described herein. As will be understood from the description below, in many important use cases, such as battlefield environments or in the aftermath of a natural disaster, networks may not be available or may be limited. In such cases, the system may be implemented to function using local resources, satellite communications or emergency networks and the functionality may adapt to such environments.

The processing platform 108 processes the sensor information and other information from the user device 102, determines risk stratification information as well as medical diagnosis and treatment option information based on machine learning technology, and provides output information to the user device to assist the user in treating the subject 104. The illustrated processing platform 108 includes a preprocessing module 118, a machine learning module 120 and a knowledge base 126. The preprocessing module 118 performs a number of functions to prepare the input data from the user device 102 for use by the machine learning module 120. In this regard, the input data may need to be processed to obtain various subject parameters. For example, video data from the user device 102 may be processed to obtain information regarding temperature, perfusion, respiratory action, or various motor functions, as described in more detail below. Audio information may be processed to determine certain vocal biomarkers such as speech patterns, tone, or rate. In addition, the input data may be annotated and classified, regions of interest or signals of interest may be selected, the data may be normalized, and features may be extracted. Thus, a variety of metadata may be associated with the input data to support the machine learning functionality.

The processing platform may be implemented on a single machine (e.g., server or computer) or multiple machines located at a single location or geographically distributed. The functionality of the platform as described herein may be replicated at each machine/location or may be distributed across machines locations. Moreover, certain functionality may be executed at the processing platform, the user device, and/or on another platform, e.g., signal information may be pre-processed at a user device for data enrichment, formatting, or compression, among other things.

The machine learning module 120 includes a training mode 122 and a live mode 124. In the training mode, training information is provided for use in developing models that can be used to generate risk stratification and medical diagnosis information. In the live mode 124, live data from a user device 102 is processed using the developed models to generate output information to provide to the user device 102. The module may implement the neural network described above for determining blood pressure based on PPG signals. Various supervised and unsupervised machine learning technologies may be employed as described in more detail below

The knowledge base 126 stores information used by and generated by the pre-processing module 118 and the machine learning module 120. This may include training data, model information, statistical data, demographic data, medical record information, and any other information that is useful in developing and executing the machine learning models. One advantage of implementing the system 100 using a centralized processing platform 108 is that, over time, a rich knowledge base accumulated over many experiences concerning different kinds of conditions for different subjects will be available to improve the accuracy of evaluations. It will be appreciated that, although the processing platform 108 is shown as a single element for purposes of illustration, the functionality of the processing platform 108 may be distributed over many machines and may be geographically distributed to improve response. For implementations of this technology where processing is either desired or required on a localized and/or individual device or platform, the technology application is updated from the centralized processing platform.

The processing platform 108 may also access certain external sources 128. Such external sources 128 may be used to gather information to assist in developing and executing the models of the machine learning module 120. This may include medical record information from medical facilities and government sources, medical records for specific subjects 104 being evaluated, demographic information, e.g., from private and government sources, modeling tools, and other information. Such information may be provided directly to the processing platform 108 or may be accessed by a user device 102 or emergency response network 130. In connection with the user device 102, emergency response network 130, processing platform 108 and external sources 128, data may be filtered or otherwise processed (e.g., anonymized, aggregated, or generalized and through use of methods such as Federated Learning) to address privacy concerns. For example, the use of particular items of information may be controlled by the user or subject 104, by policies implemented in connection with the system 100, medical facilities, or other entities, or in accordance with applicable regulations.

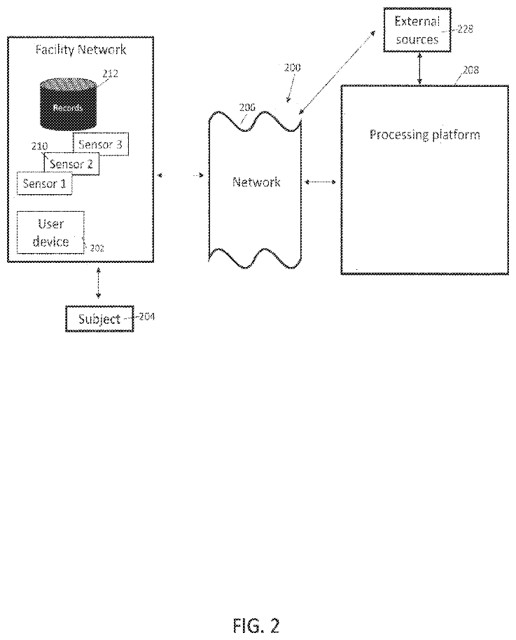

FIG. 2 shows another use case of a PreDICT system 200 in accordance with the present invention. The illustrated system 200 includes a user device 202 for use by a user in treating a subject 204, a processing platform 208, external sources 228 and a network 206 for interconnecting these various elements. The network 206, processing platform 208, and external sources 228 are generally similar to the corresponding elements described in connection with FIG. 1 and such description will not be repeated.

In this case, however, the user device 202 is implemented in connection with a facility network 214. For example, the facility network 214 may be a local area network or other network associated with a hospital, clinic, or other medical facility. The user device 202 may connect to the facility network 214 to access patient records 212, upload sensor data from the user device 202 and/or other sensors 210, and access various other network-based resources. For example, the user device may comprise a tablet computer or intelligent medical device. In this regard, information from a variety of sensors 210 may be available for transmission to the processing platform 208. Thus, a patient and medical facility may have a variety of vital sign and other information that is continuously or periodically monitored by the sensors 210 (e.g., a PPG device used to provide signals for arterial oxygen saturation, pulse rate, and/or blood pressure as described above). An application executed at the user device 202 and/or processing platform 208 may harvest sets of data from the sensors 210 on a defined schedule or on demand. It will thus be appreciated that, in the illustrated use case, the processing platform 208 may have access to a rich data set for processing and may provide correspondingly accurate and detailed reports to the user device 202 for use by skilled and expert users.

Much of the immediately preceding discussion has focused on contexts where a user is actively involved in initiating actions or inputting information. In many emergency contexts that form an important application of the present invention, the user's ability to activate sensors and input information may be limited or the user's attention may be required for other purposes. Thus, it will be appreciated that the invention may operate differently in other contexts or use cases.

To understand the functionality of the PreDICT system and the manner in which users will interface with the device, it is important to understand one of the key use cases and certain attributes of this use case, which are applicable to multiple other use cases.

USE CASE: Employment by a battlefield medic during a kinetic engagement taking care of a close and personal friend who has been badly wounded. There are multiple considerations in this scenario as to how users optimally interact with the capability: 1) physical considerations—the user's hands and/or gloves may be covered in blood, dirt, and fluid. The medic may be copiously sweating, thus impairing, or precluding interaction with the PreDICT device/interface. This may occur at night and the tactical situation may prohibit a bright touchscreen. Night vision compatible screens still encounter the problems with blood, dirt, sweat, etc. These factors make it very difficult to interact with a touchscreen or keyboard, 2) the user may be in a high emotional state and his cognitive and technical bandwidth may be consumed by taking care of the casualty, his friend. Every requirement to actively interface with the capability, other than to get exactly the information the medic needs, unnecessarily draws on his already limited bandwidth, and requires more consideration in a time-constrained problem-set. As long as the sensors are active and appropriately oriented, the PreDICT system is acquiring, processing, analyzing, and outputting information with minimal requirements for user interface. The PreDICT system can communicate this information to him through multiple means such as a screen display and/or audio information through the medic's radio headset (such as a Peltor headset). If the PreDICT system detects that the user is not optimally caring for the patient and assesses that an intervention is not necessary or that another intervention or course of action is preferable, it can “escalate its communication” with the user through various auditory and/or visual and/or tactile prompts.

If the PreDICT system requires more information to determine the desired outputs, the capability can prompt the user to enter or acquire more information. The user can then do this by adding or adjusting a sensor capability or by providing voice, touchscreen, or keyboard inputs.

Employment of multiple technical capabilities in medical and other scenarios have encountered the two key issues described above: 1) physical considerations make it difficult to interact with the device, and 2) the device places high demands on the bandwidth of the user that is otherwise required to resolve the problem at hand, which effectively makes the technical capability part of the problem. The PreDICT system will avoid these limitations and liabilities.

The bottom line is that, during the period of employment, the PreDICT system will require minimal effort or input from the user.

The PreDICT system, as a sensor and/or device and/or system and/or network, can be activated (“turned on”) actively, passively, directly, or remotely to include the ability of the PreDICT system to self-activate in response to certain signals or signal patterns, for example, if it detects gunshots, 9-1-1 is dialed, or it detects a deceleration pattern indicative of a car crash. It can also go into specific modes based on these signals.

Once activated, the PreDICT system will extract, process, and analyze data from the subject and the environment to determine what mode it needs to be in and will function accordingly. It may have one or several default settings that it will activate in response to specific signals to place it in a specific mode. Alternatively, the system may prompt the user to place it in a specific mode if it cannot extract the necessary or sufficient information or if it does not have the computational bandwidth to extract, process, and analyze the information and determine the appropriate mode.

PreDICT system users will have the ability to select certain modes and/or menus via voice, touchscreen, keyboard, or other sensor inputs. Typically, a user would select these modes outside of or in anticipation of a specific scenario or rapidly via voice or other prompts as the scenario presents. These menus will range from broad to specific. For example, broad menus cover different use case domains such as “medical” and “intentionality.” Within the “medical” heading there are multiple different chief complaints, body systems, anatomic regions, and/or subsets of pathology, etc. Within the “intentionality” heading there are multiple options such as “threat,” “truthfulness,” etc. If the user knows that they will encounter, or have a high probability of encountering, a trauma patient they may elect to place the capability in a “trauma mode.” In another scenario, and for a different domain use case, the user may place the device in “threat mode” to determine if an individual in their environment represents a threat. The purpose for preselecting modes is to preserve computational bandwidth on a PreDICT device and/or network where the capability would otherwise need to extract, process, and analyze sensor data to determine that it was in a trauma or threat scenario.

In summary, the interface functionality of the PreDICT system ranges from a default with minimal to no user interface requirements during PreDICT application to, if desired and feasible, intensive interface between user and capability. The PreDICT user interface can also be a hybrid along a spectrum between minimal interface (system is only outputting information to user) to intensive manual interface by the user into the capability. The tradeoffs between these ends of the spectrum entail a balance between the bandwidth and physical capability of the user to interface with the capability and the computational bandwidth of the PreDICT capability.

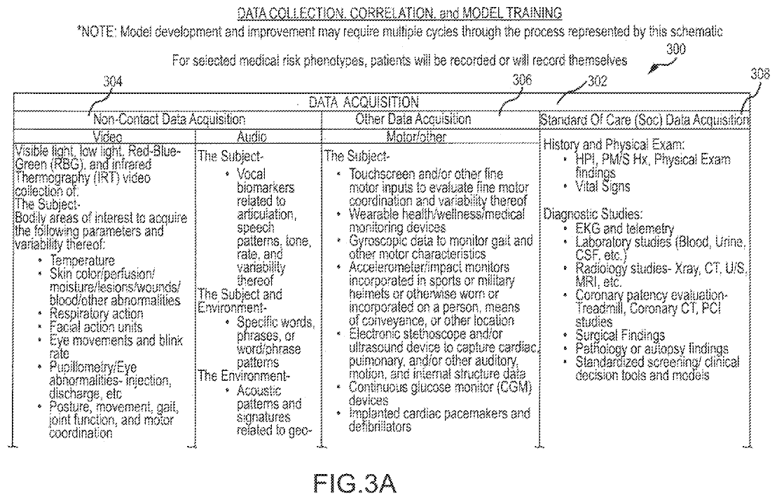

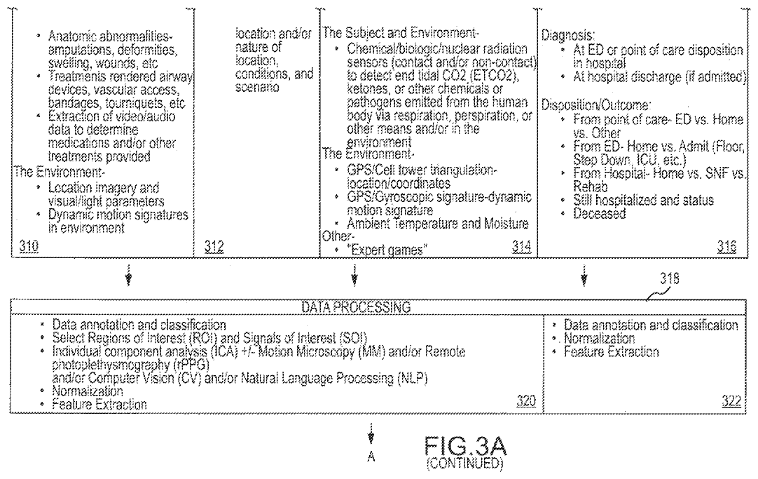

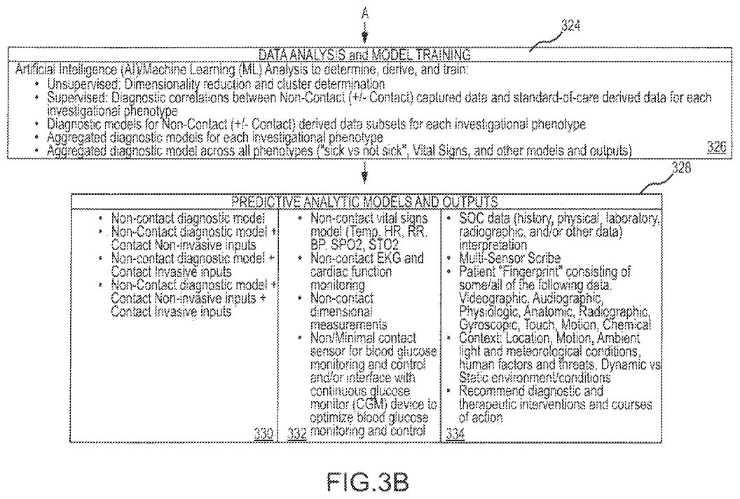

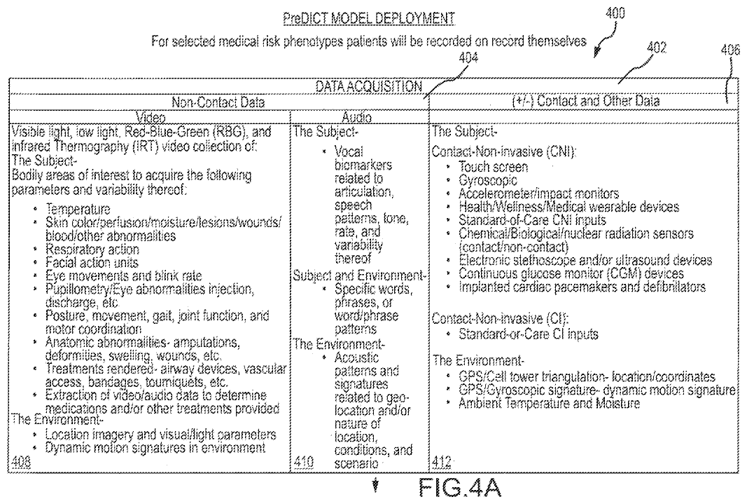

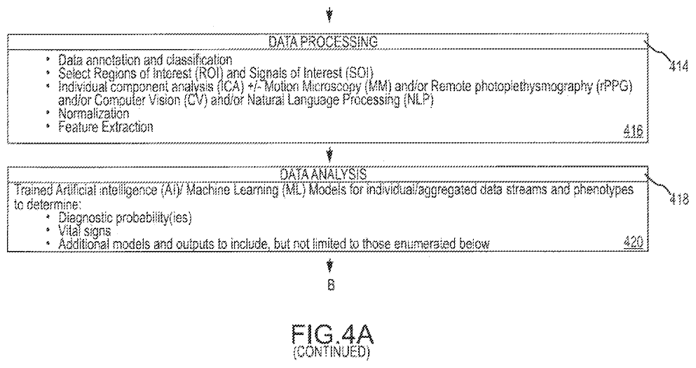

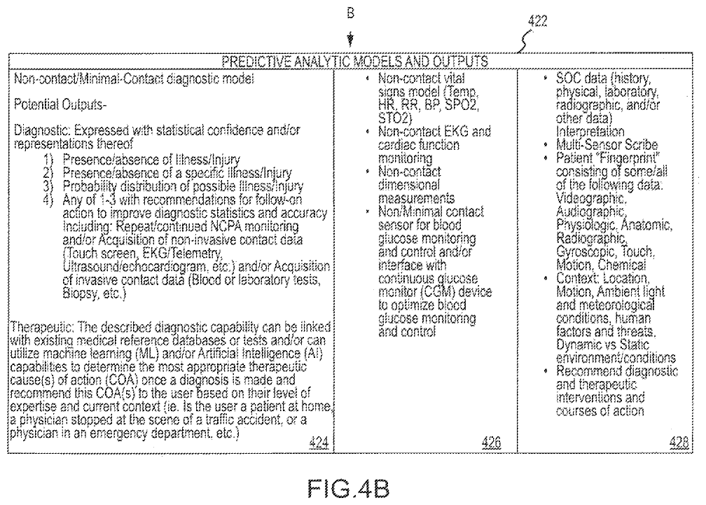

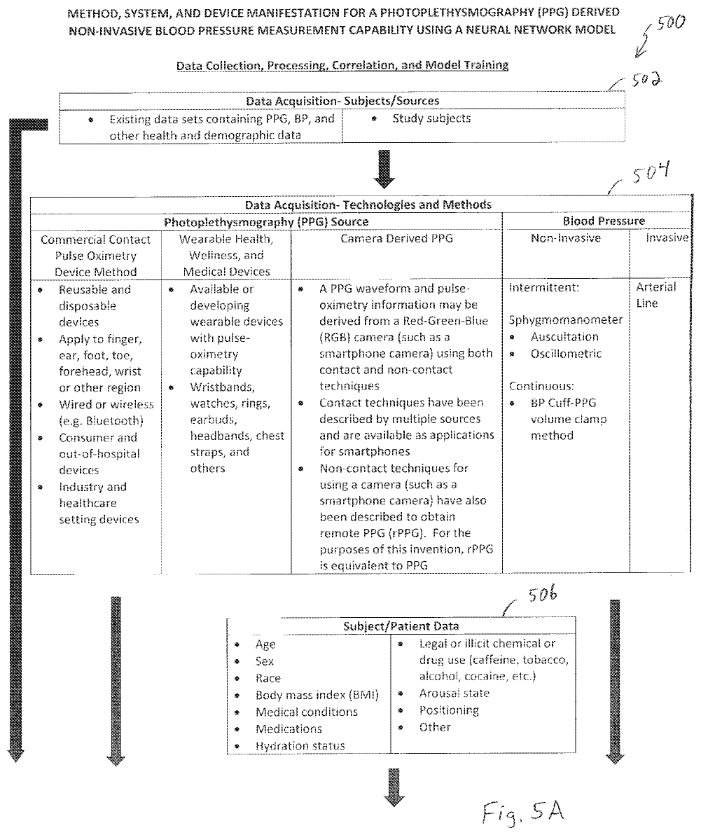

As noted above, the machine learning process is implemented in connection with a training mode and a live data mode. This may alternatively be denoted as model training and model deployment. These processes are illustrated in FIGS. 3-4.

Referring first to FIG. 3, the model training process 300 generally includes data acquisition (302), data processing (318) or preprocessing, data analysis and model training (324), and development (328) of noncontact predictive analytic models. In the illustrated process 300, data acquisition (302) involves non-contact data acquisition (304), other data acquisition (306), and standard of care data acquisition (308). The non-contact data acquisition (304) and contact data acquisition (306) processes may be implemented by users in connection with live medical evaluations or by users entering training data. In the case of users involved in live medical evaluations, the data may be entered in response to prompts of a user interface or in response to questions from a PSAP operator or another person. To illustrate, when a user accesses a processing platform of the PreDICT system, the user may be prompted to enter information regarding a current condition being evaluated, e.g., by selecting “chest pain” from a drop-down menu or otherwise describing a medical condition via a structured or free-form data entry. In response to such an input, the processing platform may execute branching logic and present additional user interface screens depending on the information entered by the user on previous screens. Such screens may prompt the user to obtain sensor information and upload the sensor information to the processing platform. For example, the user may be prompted to obtain a video clip of the subject's face and neck region and upload the video file together with an audio recording of the patient to the processing platform.

As shown, the noncontact data (304) may include video data (310) and audio data (312). The video data may be obtained using any type of camera device including but not limited to a standard webcam, a smart phone camera, Google Glass or other glasses-camera devices, GoPro® type cameras, body mounted cameras; static cameras such as security and surveillance type cameras; cameras mounted on mobile platforms such as aerial, ground based, or aquatic/maritime vehicles or autonomous or remotely operated vehicles; another red-blue-green camera; low light; and/or an infrared thermography video camera. Video data utilized by this technology may be obtained/extracted from video not expressly recorded for the purposes of applying this technology. Such cameras may be used to obtain a video recording of the head and neck region or other body areas of interests of the subject to acquire information indicative of any of the following or combinations, variability or other derivatives thereof: temperature; skin color, perfusion, or moisture; lesions, wounds, blood or other abnormalities; respiratory action; facial action unit; eye movements and blink rate; pupillometry, eye abnormalities—injection, discharge, etc.; posture, movement, gait, joint function, and motor coordination; anatomic abnormalities—amputations, deformities, swelling, wounds, etc.; treatments rendered—airway devices, vascular access, bandages, tourniquets, etc.; and extraction of audio/video to determine medications and/or other treatments provided. Such cameras may also be used to obtain information on the environment where a subject is located (or with the environment as the subject) such as location imagery; visual and light parameters; and dynamic motion signatures in the environment. The audio data, which may be obtained as an audio track accompanying a video recording and/or may be obtained separately through any capable recording device and/or derived through data processing techniques such as motion microscopy (MM), may include information indicative of vocal biomarkers for the subject and/or others in the environment related to articulation, speech patterns, tone, rate, and variability thereof. Audio data may also include specific words, phrases, and/or word phase patterns related to the subject and/or others in the environment. Audio data may also include acoustic patterns and/or signatures related to geolocation and/or the nature of the location, conditions, and scenario.

The other data (306) involves data that may be obtained via contact between the subject and a sensor and may include data on motor function or other parameters of the subject and/or environment (314). For example, the subject may be prompted to interact with materials or graphical objects presented on a touchscreen and/or to interact with other equipment to evaluate fine motor coordination and variability thereof over time. Additionally, or alternatively, sensors such as gyroscope-based instruments may be applied to the subject or embedded in devices carried by or on the subject for other purposes such as smart phones or wearable fitness devices to obtain gyroscopic data for monitoring gait and other motor characteristics. Accelerometer/impact monitors may be incorporated in sports or military helmets or otherwise incorporated on a person, means of conveyance, or other location and used to obtain impact data. As a still further alternative, wearable health/wellness/medical monitoring devices may be employed to obtain various kinds of sensor information such as pulse oximetry data (for arterial oxygen saturation or blood pressure as described above), heart rate and heart rate variability data, respiration rate, and parameters related to the autonomic nervous system. Such data acquisition may further involve chemical and/or biologic and/or nuclear radiation sensors (contact and/or non-contact) to detect end tidal CO2 (ETCO2), ketones, acetone, alcohol metabolites, or other chemicals/toxins, biologic material or organisms, or radiation emitted from the human body via respiration, perspiration, or other means and/or to detect chemicals/toxins, biologic materials or organisms, or radiation in the environment. Electronic stethoscope, doppler, and ultrasound data may be obtained to capture cardiac, pulmonary, and/or other auditory, motion, and internal structure data related to the subject. Further data on the subject may be captured using continuous glucose monitoring (CGM) devices and/or from implanted cardiac defibrillators and pacemakers. Data may also be obtained on the environment, location, and the nature of the location and environment to include ambient temperature and moisture data; global positioning system (GPS) and or cell phone tower triangulation data; and dynamic motion signatures from GPS and gyroscopic devices to determine motion parameters in multiple dimensions for scenarios such as, but not limited to, travel on ground, maritime, or aerial platforms. Lastly, data acquisition may include “expert games”; expert games are a mechanism to build or augment data sets for training machine learning and/or artificial intelligence systems and for those systems to build models. Expert games use real or hypothetical case studies of problems in domains of interest to build “games” for relevant experts. Through the “playing” of these games, key information about expert decision making and the problem-sets posed by the “games” can be extracted to create data sets for machine learning and/or artificial intelligence analysis, learning, and modeling. The PreDICT system will use expert games to augment training and functionality for application to multiple domain scenarios. Expert games will particularly apply when training and modeling high-consequence, low frequency events.

Sensor platforms may include fixed camera and/or audio recording or other devices for the purpose of obtaining input data related to the diagnostic and/or predictive capabilities of this capability or fixed sensors not explicitly for the purposes of this capability, such as surveillance cameras. Sensor platforms may also include human or vehicle (to include ground, air, and maritime platforms both manned, unmanned, and autonomous) mounted or transported sensors. Remotely piloted and/or autonomous ground, air, and maritime vehicles will provide important platforms for PreDICT as sensor platforms and/or as network nodes for PreDICT capability and/or by using PreDICT capability as the decision-making application to guide the functionality of the platform as in the case of autonomous systems.

The standard of care (SOC) data (308) may be obtained from the subject, the user, patient records of the subject, patient records from a medical facility, peer-reviewed literature, government databases, other third-party databases, and other sources. Examples (316) of such data include records of the subject's medical history and physical exam data such as history of present illness/injury (HPI) data, past medical and surgical (PM/S Hx) to include allergies and medications, physical exam findings and vital signs, possibly including electronic stethoscope data. In addition, the data may be obtained from diagnostic studies such as electrocardiogram (EKG) and telemetry, laboratory studies (blood, urine, cerebral spinal fluid (CSF), etc.), Radiology studies (e.g., x-ray, computed tomography (CT), ultrasound (U/S), and magnetic resonance imaging (MRI)), coronary patency evaluation (e.g., treadmill stress test, coronary CT, and percutaneous coronary intervention (PCI) studies), cardiac catheterization, surgical findings, pathology and autopsy findings, electroencephalogram (EEG), and standardized screening and clinical decision tools and models. The standard of care data (308) may further include diagnoses such as those made at emergency department (ED), clinic or point-of-care disposition, in-hospital diagnoses and diagnoses made at hospital discharge (if admitted). Finally, the data (308) may include disposition/outcome data from the point-of-care (ED vs. home vs. other), from the ED (home vs. admit—floor, step down, ICU, etc.), and/or from the hospital (home vs. SNF vs. rehab). The disposition/outcome information may also include status information such as whether the subject is still hospitalized and their current status or whether the subject is deceased. Standard of care data and other medical data may also be acquired from other treatment environments and paradigms (e.g., non-clinic, non-emergency department, non-hospital based under some standard conditions) such as deployed military medical treatment facilities, humanitarian medical programs, medical disaster response scenarios, austere medical events, or programs, and/or emergency medical services

The data processing (318) involves pre-processing of input data so that it is suitable for use in a machine learning process. As noted above, this may involve processing raw inputs to obtain the desired parameters. For example, infrared camera data may be processed to obtain temperature information and variations thereof or video files may be analyzed to obtain information regarding facial or eye movements. Such input information or parameter information may be further supplemented to assist in processing by the machine learning module. For example, noncontact data (304) and/or contact data (306) may be processed (320) to annotate and classify the data, to select regions of interest and signals of interest for further processing, to perform individual component analysis for example with or without motion microscopy and/or remote photoplethysmography and/or computer vision, and/or natural language processing, to normalize the data to facilitate comparisons, and to perform feature extraction. The standard of care data (308) may be processed to annotate and classify the data, to normalize the data, and to perform feature extraction among other things.

The data analysis and model training (324) involves processing the training data to develop models for use in analyzing live data. In the illustrated process 300 this involves using artificial intelligence/machine learning analysis to determine, derive, and train (326) the models. Artificial intelligence techniques may include, but are not limited to, neural network techniques. A variety of machine learning processes may be used in this regard including unsupervised machine learning for dimensionality reduction and cluster determination; supervised machine learning to develop diagnostic correlations between noncontact and/or contact capture data and standard of care derived data for each investigational phenotype; developing diagnostic models for noncontact and/or contact derived data subsets for each investigational phenotype; developing aggregated diagnostic models for each investigational phenotype; and developing aggregated diagnostic models across all phenotypes (sick vs. non-sick and vital signs) among other processes.

The results of the data analysis and model training (324) is the development of noncontact predictive analytic models (328). These include diagnostic models (330), noncontact models (332), and other outputs (334). The diagnostic models (330) may further include standalone non-contact diagnostic models, non-contact diagnostic models plus contact non-invasive inputs, non-contact diagnostic models plus contact invasive inputs, non-contact diagnostic models plus contact noninvasive inputs plus contact invasive inputs. The noncontact models (332) may include non-contact vital signs models, including temperature, heart rate (HR), respiratory rate (RR), blood pressure (BP), pulse oximetry (SPO2), tissue oxygen saturation (STO2); non-contact electrocardiogram (EKG) (or functional EKG equivalent) and cardiac function monitoring; non-contact dimensional measurements (e.g., video and/or sonographically derived measurements to determine the size and volume of anatomic, pathologic, or other human and non-human/non-living structures or entities); and a non/minimal contact sensor for blood glucose monitoring and control and/or interface with a continuous glucose monitoring (CGM) device to optimize blood glucose monitoring and control. The other outputs (334) may include standard of care (SOC) data (history, physical, laboratory, radiographic, and/or other data) interpretation; a “Multi-Sensor Scribe” that converts data streams into written, graphic, or other documentation formats for direct integration into existing electronic medical records (EMR) systems or other purposes; a “fingerprint” of a subject or environment including some or all of video, audio, pathologic, physiologic, anatomic, radiographic, gyroscopic, touch, motion, and chemical data; contextual models of the environment to guide decision making that include location, motion, ambient light and meteorological conditions, human factors and threats, and assessment of whether the context is static versus dynamic; and recommendations on diagnostic and therapeutic courses of action.

FIG. 4 illustrates a PreDICT model deployment process 400. In particular, the process 400 is illustrated with respect to four diagnostic models and additional models developed by the machine learning training process. The illustrated process 400 is initiated by data acquisition (402). In this case, the data acquisition (402) generally corresponds to the noncontact data acquisition (404) and contact data acquisition (406) described above in connection with FIG. 3. Indeed, it is anticipated that live data will also be processed through the model training process to further develop the models. Thus, the noncontact data (404) may include video data (408) and audio data (410), and the contact data (406) may include motor inputs and standard of care contact-non-invasive (CNI), and contact-invasive (CI) inputs (412) as described above. In addition, the illustrated data processing (414) may include various preprocessing functions (416) as described above in connection with FIG. 3.

However, in this case, the data analysis (418) involves deploying the trained machine learning models (420) with respect to individual or aggregated data streams and phenotypes to determine diagnostic probabilities, vital signs, and other outputs. Specifically, in the case of deploying the non-contact/minimal-contact predictive analytic models (422) with respect to live data involves deploying a non-contact/minimal-contact diagnostic model (424), deploying another non-contact model (426), and/or providing other outputs (428). The potential outputs of the diagnostic model (424) may include diagnostic and therapeutic outputs. The diagnostic output may be expressed with statistical confidence and/or representations thereof with respect to: 1) the presence or absence of illness or injury; 2) the presence or absence of a specific illness or injury; 3) a probability distribution for particular diagnoses; and any of items 1-3 with recommendations for follow-on action to improve diagnostic statistics and accuracy. Such follow-on actions may include repeat or continued non-contact predictive analytic (NCPA) monitoring and/or acquisition of noninvasive contact data (touchscreen, EKG/telemetry, ultrasound/echocardiogram, etc.) and/or acquisition of invasive contact data (laboratory tests, biopsy, etc.).

For the therapeutic output, the described diagnostic capability can be linked with existing medical reference databases or texts and/or can utilize machine learning and/or artificial intelligence, such as neural network capabilities, to determine the most appropriate therapeutic courses of action once a diagnosis is made and recommend this course of action to the user based on their level of expertise and current context. In this regard, the therapeutic output may consider whether the user is a patient at home, a physician stopped at the scene of a traffic accident, a physician in an emergency department, etc.

The other models and outputs (426) may include a non-contact vital signs model (temp, HR, RR, BP, SPO2, STO2), a non-contact EKG and cardiac function monitoring model, a non-contact dimensional measurements model, and a non/minimal contact sensor for blood glucose monitoring and control and/or interface with a continuous glucose monitoring (CGM) device to optimize blood glucose monitoring and control. The other outputs (428) May include standard of care (SOC) data (history, physical, laboratory, radiographic, and/or other data) interpretation; a multi-sensor scribe that converts data streams into written, graphic, or other documentation formats for direct integration into existing electronic medical records (EMR) systems or other purposes; a “fingerprint” of a subject or environment including some or all of video, audio, pathologic, physiologic, anatomic, radiographic, gyroscopic, touch, motion, and chemical data; a contextual model of the environment that includes location, motion, ambient light and meteorological conditions, human factors, threats, and a measure of static versus dynamic conditions, and other parameters to guide contextual decision making on treatments and courses of action; and recommendations on diagnostic and therapeutic courses of action

The present invention is this applicable with respect to a variety of conditions and in a variety of contexts as set forth below.

Examples of Medical Conditions and Contextual Circumstances where technology provides utility: (Note: “Utility” refers to any of “ruling in”, “ruling out”, decreasing time to diagnosis, decreasing required interventions to arrive at diagnosis, decreasing cost, monitoring for deterioration/improvement, etc.)

Medical Conditions: Including but not limited to:
Neurologic:
   Stroke (Cerebrovascular Accident (CVA)) and/or Transient Ischemic Attack (TIA)
   Traumatic Brain Injury (TBI)
   Spinal cord injury, compression, ischemia, infection
   Altered mental status
   Dementia vs. Delirium
Psychiatric/Mental Health/Developmental Conditions:
   Suicidality or risk for self-harm
   Homicidality or risk of harm to others
   Depression
   Mania
   Delirium
   Post Traumatic Stress Disorder (PTSD)
   Autism
Cardiopulmonary/Chest Pain:
   Heart Attack (Acute coronary syndromes (ACS))
   Dys-/arrhythmia
   Aortic Dissection
   Pulmonary Embolism (PE)
   Pneumothorax (PTX)
   Esophageal Rupture
   Pneumonia
   Asthma/COPD
   Congestive Heart Failure (CHF)
Cardiovascular:
   Blood pressure monitoring
   Hypertensive urgency/emergency
Pre-/Shock States:
   Distributive
   Hypovolemic
   Cardiogenic
   Obstructive
   Dissociative
   Resuscitation monitoring
Infectious Disease:
   Systemic infectious processes (i.e. Sepsis, COVID-19, etc.)
   Localized infectious processes (i.e. Necrotizing fasciitis, cellulitis, pyelonephritis, etc.)
Intraabdominal and OB/GYN Processes:
   Appendicitis, Cholecystitis, Diverticulitis, Abdominal aortic aneurysm (AAA), etc.
   Ectopic pregnancy
   Ovarian torsion or cyst rupture
Ischemic Processes (not already mentioned):
   Embolic processes resulting in ischemic limb or other organ/region
   Testicular torsion
Musculoskeletal:
   Joint injury such as sprain, dislocation, or meniscus or labral tear
   Bone fracture
Trauma:
   Blunt
   Penetrating
   Burn
Toxicology:
   Toxidromes
   Intoxication
Metabolic and Endocrine disorders (e.g. Diabetes, glucose monitoring)
Malignancies/Cancer

Contextual Circumstances:
Conventional medical settings: Doctor's office, Emergency Department, In hospital
Mass casualty events/incidents (aka. MASCAL, MCI)—Triage, risk-stratification, diagnosis
Austere and/or resource constrained environments
Pre-hospital (EMS)
Out of hospital (laypersons)
Telemedicine
Disease surveillance
Time challenged diagnoses (e.g., TBI)
Out of hospital monitoring
Military applications and combat settings

### Outputs:

The PreDICT system not only recommends what intervention a patient requires but also the logistics and sequencing of that intervention by processing not only information about the patient but also information about the risk-context surrounding the patient and their illness/injury. Several examples are below.
   Example 1—The PreDICT system determines that a trauma patient requires endotracheal intubation because of an increasing inability to protect his airway. However, the PreDICT system also determines that the patient is hypotensive and has a probable pneumothorax. The PreDICT system determines that positive pressure ventilation from endotracheal intubation will cause immediate decompensation by: 1) Increasing intrathoracic pressure in the setting of hypotension, which will decrease blood return to the heart via the vena cava, decrease cardiac preload, and decrease cardiac output and 2) It will convert the pneumothorax to a tension pneumothorax from the positive pressure ventilation, further increasing intrathoracic pressure and accelerating decompensation. Following this analysis PreDICT recommends an intervention sequence of: Step 1) Simultaneous chest tube placement and rapid infusion of 1 unit of whole blood, Step 2) Endotracheal intubation once Step 1 complete and blood pressure achieves a minimum of XYZ/xyz.
   Example 2—The PreDICT system determines that a patient at Hospital A with chest pain is experiencing an ST elevation myocardial infarction (STEMI) and requires a cardiac catheterization to relieve the coronary artery obstruction. Hospital A does not have this capability, but Hospital B does. This is a time-constrained medical problem-set (“time is myocardium”) and the patient's probability of survival and optimal future cardiac function is inversely related to the time to the procedure. The patient can be transported by helicopter or ground ambulance. The ambulance can have the patient loaded and depart in 10 minutes. It will take 5 minutes to get the patient to the cardiac catheterization lab at Hospital B once the patient arrives. The helicopter can have the patient loaded and depart in 30 minutes. It will take 10 minutes to get the patient to the cardiac catheterization lab at Hospital B once the patient arrives. The PreDICT system can evaluate historical transport data and real-time air and ground traffic data to determine that transport by helicopter will place the patient in the cardiac catheterization lab at Hospital B twelve minutes faster than transport by ground ambulance at this time of day due to heavy traffic volumes. Alternatively, the PreDICT system may recommend that the patient should be administered thrombolytic treatment for the STEMI at Hospital A because the transport time to Hospital B by either mode is prohibitively long given the patient's STEMI and the time since onset. (Thrombolytics are a “second line” treatment for STEMI if the patient cannot undergo cardiac catheterization within a recommended time window.)

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1. A method for use in measuring blood pressure of subjects, comprising:
obtaining first and second processed signal information, said first signal information corresponding to first signals of one or more first subjects obtained using a photoplethysmography (PPG) device and said second signal information corresponding to second signals of said first subjects obtained using a blood pressure device different than said PPG device;
developing a neural network model for obtaining blood pressure information based on signals of said PPG device using said first and second processed signal information; and
validating said neural network model by comparison of blood pressure measurements obtained using said model to blood pressure measurements obtained using other blood pressure measurement devices.

2. The method as set forth in claim 1, wherein said obtaining comprises first obtaining said first signals by using said PPG device on said first subjects and second obtaining said second signals by using said blood pressure device on said first subjects.

3. The method as set forth in claim 2, wherein said step of first obtaining comprises operating one of a purpose-built pulse oximetry device, a wearable health device, and a smart phone.

4. The method as set forth in claim 2, wherein said step of second obtaining comprises operating one of a pneumatic cuff device for measuring blood pressure and an invasive, continuous blood pressure measuring device.

5. The method as set forth in claim 1, wherein said obtaining comprises preprocessing said first and second signals to get said first and second processed signal information, respectively.

6. The method as set forth in claim 5, wherein said preprocessing comprises one of noise reduction, normalization, and segmentation of said first and second signals.

7. The method as set forth in claim 5, wherein said preprocessing comprises feature identification and extraction with respect to said first and second signals.

8. The method as set forth in claim 5, wherein said preprocessing comprises time shifting at least one of said first and second signals for alignment of waveforms.

9. A system for use in measuring blood pressure of subjects, comprising:
a data storage unit for storing first and second processed signal information, said first signal information corresponding to a first signals of one or more first subjects obtained using a photoplethysmography (PPG) device and said second signal information corresponding to second signals of said first subjects obtained using a blood pressure device different than said PPG device;
a processing module for accessing said first and second processed signal information from said data storage unit and developing a neural network model for obtaining blood pressure information based on signals of said PPG device using said first and second processed signal information; and
validating said neural network model by comparison of blood pressure measurements obtained using said model to blood pressure measurements obtained using other blood pressure measurement devices.

10. The system as set forth in claim 9, further comprising said PPG device for obtaining said first signals from said first subjects and said blood pressure device for obtaining said second signals from said first subjects.

11. The system as set forth in claim 10, wherein said PPG device comprises one of a purpose-built pulse oximetry device, a wearable health device, and a smart phone.

12. The system as set forth in claim 10, wherein said blood pressure device comprises one of a pneumatic cuff device for measuring blood pressure and an invasive, continuous blood pressure measuring device.

13. The system as set forth in claim 9, wherein said processing module is operative for preprocessing said first and second signals to get said first and second processed signal information, respectively.

14. The system as set forth in claim 13, wherein said preprocessing comprises one of noise reduction, normalization, and segmentation of said first and second signals.

15. The system as set forth in claim 13, wherein said preprocessing comprises feature identification and extraction with respect to said first and second signals.

16. The system as set forth in claim 13, wherein said preprocessing comprises time shifting at least one of said first and second signals for alignment of waveforms.

17. A method for use in measuring blood pressure of subjects, comprising:
providing a neural network model for obtaining blood pressure information based on signals from a photoplethysmography (PPG) device;
operating said PPG device to obtain a signal from a first subject;
preprocessing said signal to obtain preprocessed signal information for use in said neural network model;
applying said neural network model to said preprocessed signal information to obtain blood pressure information; and
outputting said blood pressure information.

18. The method of claim 17, further comprising using said neural network model to define an application program interface (API) for deployment in a system to facilitate remote acquisition of said blood pressure information.

19. The method as set forth in claim 17, wherein said preprocessing comprises one of noise reduction, normalization, and segmentation of said signal.

20. The method as set forth in claim 17, wherein said preprocessing comprises feature identification and extraction with respect to said signal.

21. The method as set forth in claim 17, wherein said preprocessing comprises time shifting said signal for alignment of waveforms.

22. The method as set forth in claim 17, wherein said PPG device comprises one of a purpose-built pulse oximetry device, a wearable health device, and a smart phone.

23-33. (canceled)
